(19) United States
(12) Patent Application Publication
(10) Pub. No.: US 2014/0088299 A1
(43) Pub. Date: Mar. 27, 2014

(54) METHOD OF EXTRACTING PHENOLIC FRACTIONS OF EXTRA VIRGIN OLIVE OIL

(71) Applicants: Timothy A. Klapish, Raleigh, NC (US); Thomas Gutierrez, Manchester, NH (US); C. Chad Harrell, Raleigh, NC (US); M. Christine Hillhouse, Apex, NC (US)

(72) Inventors: Timothy A. Klapish, Raleigh, NC (US); Thomas Gutierrez, Manchester, NH (US); C. Chad Harrell, Raleigh, NC (US); M. Christine Hillhouse, Apex, NC (US)

(73) Assignee: PhytoChem Pharmaceuticals, Inc., Cary, NC (US)

(21) Appl. No.: 14/002,920

(22) PCT Filed: Mar. 2, 2012
(86) PCT No.: PCT/US12/27365

§ 371 Date: Nov. 4, 2013

Related U.S. Application Data
(60) Provisional application No. 61/448,265, filed on Mar. 2, 2011.

(51) Int. Cl. C07H 17/07; C07C 37/00; C07D 309/32

(52) U.S. Cl. CPC C07H 17/07; C07D 309/32; C07C 37/004
USPC 536/18.1; 549/420; 568/749

(57) ABSTRACT

The present invention relates to isolating phenolics from extra virgin olive oil (EVOO) having a low triglyceride and non-polar content. The method includes an ethanol/water extraction with a heptane wash

This application claims priority of U.S. provisional application No. 61/448,265 filed on Mar. 2, 2011 and included herein in its entirety by reference.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

1. Field of the Invention

The present invention relates to the isolation phenolic compounds with low triglyceride and non-polar content from extra virgin olive oil (EVOO). In particular, it relates to a liquid extraction process for the isolating of desirable phenolic compositions from EVOO.

2. Description of Related Art

A number of vegetable oils are known to be beneficial in the diet when compared to animal based fats. Oils, such as olive oil and especially extra virgin olive oil, are known to be cardio protective and certain known fractions have been shown to have medicinal benefits.

Olive oil, especially EVOO, is known to be low in saturated fats and provides its health benefits due to phenolic content or fatty acid profile of the olive oil being responsible for cardio-protective benefits. Olive oil contains monounsaturated fats, such as oleic acid, and has antioxidants such as phenolics, Vitamin E, carotenoids, and oleuropein. This fatty acid, as well as linoleic and others, make up the fatty acid portion of olive oil. The rancidity and taste grading of olive oil is tied to the presence of free, esterified oleic acid with grades of olive oil being made based on higher grades having a lower free oleic acid content. High acid content oils are frequently refined to chemically neutralize these compounds, though higher grade oils require that they cannot contain neutralized triglycerides and instead must just contain lower amounts or triglycerides.

Much research has been done into fractionating olive oil (as well as other beneficial vegetable oils) and testing the results. The two main methods of separation or removing unwanted components involve filtration or a liquid extraction process. While these methods of separation result in isolation of useful compounds, they frequently do not separate out other undesirable compounds. For example, solvent liquid separation is known to separate based on polarity and thus, polar compounds which are undesirable accompany the desired components during separation. These methods do not single out triglycerides or other particular compounds. In addition, processes for isolating compounds from EVOO tend to be costly and not very environmentally friendly especially when scaled up to commercial volumes.

In U.S. Pat. No. 5,089,139 to Asbeck issued Feb. 18, 1992 there is disclosed a method for refining virgin olive oil in which the virgin olive oil is filtrated by microfiltration techniques. This method avoids multi-stage filtration techniques and use of a filtration aid. It is only capable of filtering out impurities below a certain concentration. In U.S. Pat. No. 6,849,770 to Guxman et al. issued Feb. 1, 2005, there is a method described for obtaining purified hydroxytyrosol from products and by-products derived from the olive tree by means of a two step chromatographic treatment. Other articles, and the like, also describe the isolation of organic compounds from olive oil waste.

Triglycerides are particular glycerides wherein the glycerol has been esterified with three fatty acids. They are a significant component in both animal fats and vegetable oils. The triglycerides present in vegetable oils, such as olive oil, are linked to disease, such as atherosclerosis and, increased risk of heart disease and stroke. They have also been implicated in diabetes, pancreatitis, renal disease, and certain forms of primary hyperlipidemias. High triglyceride levels have also been associated with obesity.

Many organizations, such as the American Heart Association, and most experts recommend taking affirmative action to reduce triglyceride levels. One of those methods is based on the fact that reduced consumption of triglycerides can aid in a healthy lifestyle with lower triglycerides. It is known that even when triglyceride levels in food are high in infants and children, it can lead to higher triglyceride levels and hypercholesteremia in adulthood. However, even healthy oils, such as olive oil, contain a certain amount of triglycerides. This is true even though extra virgin olive oil is lower in oleic acid than even other grades of olive oil. However, the lower the triglyceride levels in olive oil and other oils, the better the oils are considered for the diet.

### BRIEF SUMMARY OF THE INVENTION

The present invention relates to methods for isolating a fraction of phenolic material from extra virgin olive oil that reduces the amount of triglycerides and other non polar lipids present with the phenolic extract than other known extraction procedures for extra virgin olive oil.

Accordingly, one embodiment of the present invention comprises a method for isolating phenolics from EVOO, wherein the isolated phenolics have a low triglyceride and non-polar content comprising:

a) selecting a desired quantity of EVOO for extraction;

b) extracting the EVOO a plurality of times with an ethanol/water solution;

c) isolating the ethanol/water solution after each extraction;

d) rinsing the ethanol/water solution with a heptane solution;

e) isolate the ethanol/water solution from the heptane; and

f) evaporate the ethanol/water solution to remove the phenolics from the solution.

In another embodiment of the present invention there is a phenolic extract of EVOO having low triglycerides and non-polar content manufactured by the method above.

### BRIEF DESCRIPTION OF THE DRAWINGS

FIG. 1 is a flow chart of the method of the present invention.

FIG. 2 is a TLC plate analysis of a phenolic extract sample compared to a lipid mixture standard.

### DETAILED DESCRIPTION OF THE INVENTION

While this invention is susceptible to embodiment in many different forms, there is shown in the drawings and will herein be described in detail specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings. This detailed description defines the meaning of the terms used herein and specifically describes embodiments in order for those skilled in the art to practice the invention.

### DEFINITIONS

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

The terms “about” and “essentially” mean ±10 percent.

Reference throughout this document to “one embodiment”, “certain embodiments”, and “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

The drawings featured in the figures are for the purpose of illustrating certain convenient embodiments of the present invention, and are not to be considered as limitation thereto. Term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

As used herein the term “EVOO” or “extra virgin olive oil” is used to define the first pressings of olives to remove the edible oil therefrom. The oil can be from any source noting that different sources present different profiles of triglycerides and other components capable of being fractionated. EVOO from countries such as the US, Spain, Italy, France, Israel, Middle Eastern countries, and the like. are all contemplated within the scope of the invention.

As used herein “phenolics” are well known components of EVOO. Olive oil phenolic constituents have been shown, in vitro, to be endowed with potent biological activities including, but not limited to, an antioxidant action. To date, there is no information on the absorption and disposition of such compounds in humans. It appears that polar olive oil phenolics, namely tyrosol and hydroxytyrosol, are dose-dependently absorbed in humans after ingestion and that they are excreted in the urine as glucuronide conjugates. Furthermore, an increase in the dose of phenolics administered increases the proportion of conjugation with glucuronide. Animal and in vitro studies suggest that all olive oil phenols are effective antioxidants. The most abundant phenols in olive oil are the nonpolar oleuropein- and ligstroside-aglycones and the polar hydroxytyrosol and tyrosol.

As used herein “isolating” refers to separating one or more of the component compounds in the oil that make up the mixture that exists in the EVOO, or separating the mixtures used in the isolation of the phenolics of the present invention. Since all EVOO contain a number of component compounds, the oil can be separated into a large number of fractions if desired. However, if one desires only a particular fraction or compound, the number might only be two or three fractions or the like when separating the oil. In fact, how many fractions depends on which component is to be separated and where it comes off of the solid phase during the separation process. For example, in the example for isolating triglycerides in extra virgin olive oil, (there are a number of different triglycerides including oleic acid), it is the second fraction that contains the triglycerides necessitating three fractions to get the triglycerides isolated from the remainder of the olive oil components. In the present invention the isolated fraction will be low in triglycerides and low in non-polar phenolics and contain a very high percentage of polar isolated phenolics.

As used herein the phrase “extracting the EVOO” refers to the process of mixing an ethanol/water solution with the EVOO in a suitable container and mixing, agitating, or the like, the mixture until compounds in the EVOO become dissolved in the ethanol/water mixture. In general, the amount of water added to the ethanol to make the ethanol/water solution is about 10 to about 40% on a w/w basis. In one embodiment, the water is about 20% on a w/w basis. In the present process the EVOO is extracted at least two times and in other embodiments 3, 4, or more, as needed to efficiently extract the EVOO as much as possible. The ethanol/water solution with dissolved components is then separated from the EVOO. Separation can be done by letting the layers settle due to their immiscibility and using standard techniques like separation funnels, pipettes, or the like to remove the EVOO and leave the ethanol/water solution. While one skilled in the art reading the description herein would understand how much of the ethanol/water solution to mix with EVOO to perform such an extraction, in one embodiment about 200 mL of the solution is mixed with about 400 grams of EVOO for each extraction.

The plurality of extractions can be combined for further processing or in one embodiment combined and processed at the same time. By combining the extractions, only one liquid needs to be taken further in the process. In other embodiments, ethanol/water extractions from different and numerous extractions can be combined even from different EVOO's at different times, volumes, and the like. The single or collection of ethanol/water extraction solutions are then combined with heptane and extracted again. While one skilled in the art could easily determine an optimum amount of heptane to combine with the extraction solutions in one embodiment about 200 mL of heptane are combined with every 600 mL of the extraction solution. The heptane layer is then separated from the extraction solution. In one embodiment the ethanol/water extraction solutions is separated by allowing the layers to separate and using standard separation techniques.

The final product is now dissolved in the heptane extraction solution. The product can be removed by any means but one embodiment of the invention, the heptane extracted solution is evaporated, for example, using rotary evaporation, though any evaporation method is certainly within the skill in the art especially during a commercial scale up process. The evaporation process is aided by the additional process of adding one or more aliquot portions of pure ethanol solution during the evaporation process. The product can be obtained by evaporation to dryness with one or more ethanol washings. In one embodiment the ethanol washing is to add about 20 mL of ethanol for every 50 mL of evaporation solution left. In one embodiment the washing is done twice.

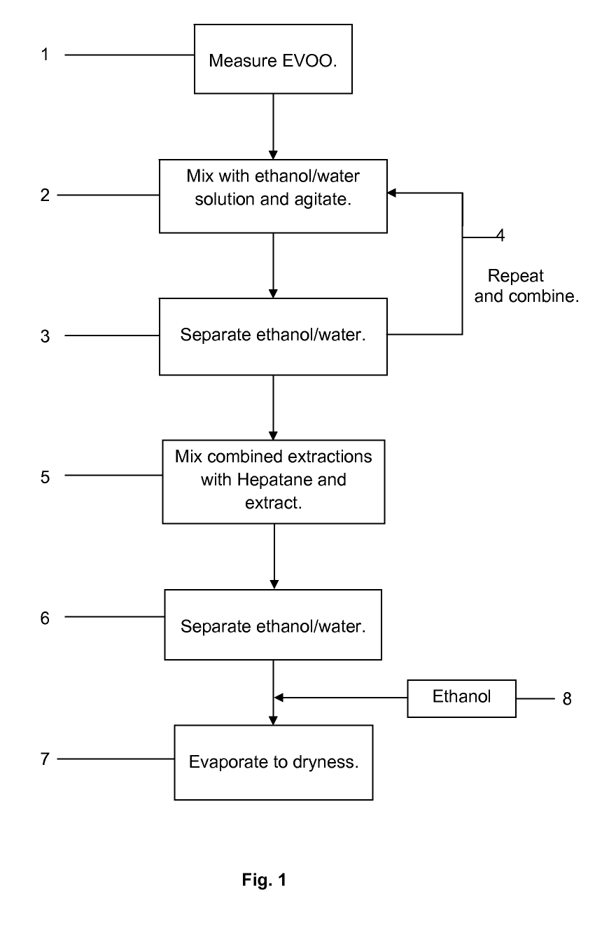

Now referring to FIG. 1 which is a flow chart of an embodiment of the present invention. A sample of EVOO (e.g. Spanish EVOO) is measured 1 and placed into a suitable container for extraction. The EVOO is mixed with a suitable solution of ethanol/water (e.g. 20% water) and agitated 2 to extract components of the EVOO. The ethanol/water solution is then separated 3 from the EVOO. The EVOO is mixed with one or more additional ethanol/water solutions and extracted and the extractions combined 4 to form a single ethanol/water solution. The combined extraction is then mixed with heptane and extracted 5 by sufficient agitation to remove triglycerides and non-polar phenolics. The heptane is then separated from the ethanol/water extraction 6 and the heptane discarded. The separated ethanol/water solution is then evaporated to dryness 7 with additional aliquots of ethanol 8 added during the evaporation process to aid in the process. This addition of ethanol during the evaporation process aids in removing all the water from the extracts due to a change in the azeotrope. The product of this process is the product claimed by the process herein.

### EXAMPLE

The EVOO extraction process is a multi-step process that consists of both extraction and evaporation steps on the EVOO sample. First weigh out 400 grams of oil into a 1 L bottle and record the weight. Then add 200 mL of a suitable solution of ethanol/water (e.g. 20% water) to the 1 L bottle and shake for 15 minutes. Let the sample reach equilibrium to form two different layers within the solution. Once the layers have formed remove the ethanol:water layer from the sample container and place in a separate 1 L bottle. Repeat this process 3-4 times to obtain a total collected volume of 600 mL of ethanol:water layer extract. The next step is to add 200 mL of heptane to the 600 mL ethanol:water collection and mix well. After the solution has been mixed allow the solution to reach equilibrium and form two layers. Once the two layers have separated remove the ethanol:water layer and place in a separate container. The ethanol:water layer that was collected is then evaporated using a rotary evaporator. Once the solution is at a volume of <20 mL, add 50 mL of ethanol solution to the container. Then continue to evaporate the solution with the rotary evaporator. Repeat this process until the solution is evaporated to <5 mL. Then transfer the extract to a pre-weighed vial and rinse the round bottom rotary evaporator flask with 10 mL of ethanol into vial. The final volume of extract is then placed into a speed vacuum system until dryness and the final weight of the extract is recorded.

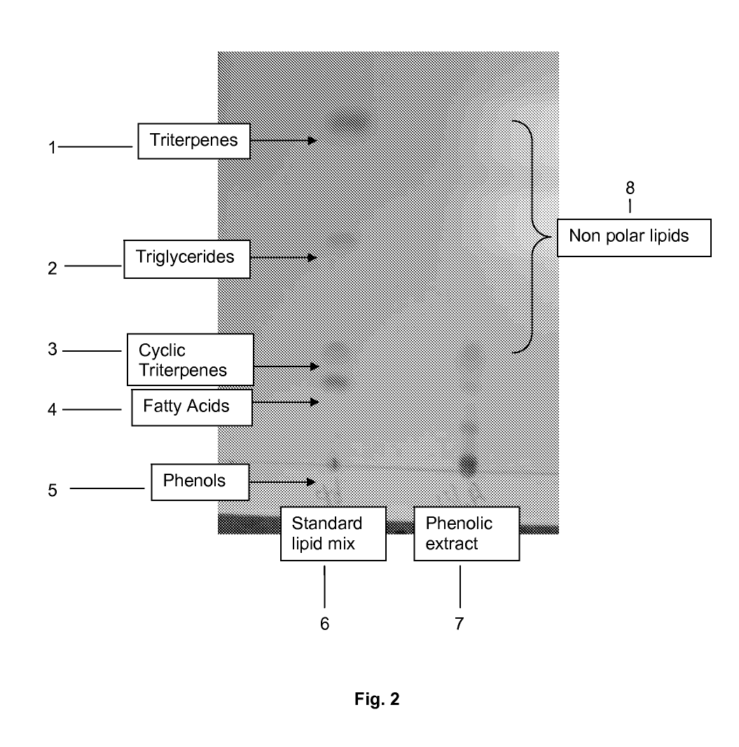

FIG. 2 shows the results of the present invention by way of a TLC Plate of a phenolic sample of the present invention compared to a lipid mixture standard. Each spot is identified and compared.
The spots are as follows:
1—Triterpenes
2—Triglycerides
3—Cyclic Triterpenes
4—Fatty Acids
5—Phenols
6—Standard lipid mix
7—Phenolic extract
8—Non polar lipids

What is claimed is:

1. A method for isolating phenolics from EVOO, wherein the isolated phenolics have a low triglyceride and non-polar content comprising:
a) selecting a desired quantity of EVOO for extraction;
b) extracting the EVOO a plurality of times with an ethanol/water solution;
c) isolating the ethanol/water solution after each extraction;
d) rinsing the ethanol/water solution with a heptane solution;
e) isolate the ethanol/water solution from the heptane; and
f) evaporate the ethanol/water solution to remove the phenolics from the solution.

2. A method according to claim 1 wherein the plurality isolated solutions of step c) are combined before step d).

3. A method according to claim 1 wherein the evaporation is carried out by a method selected from the list comprising rotary evaporation and speed vacuum evaporation.

4. A method according to claim 1 wherein the ethanol comprises about 50 to 90 percent of the ethanol/water solution.

5. A method according to claim 4 wherein the ethanol comprises about 80 percent of the ethanol/water solution.

6. A method according to claim 1 which further comprises the addition of further ethanol/water solution to the isolated solution in step e) during the evaporation process.

7. A phenolic extract of EVOO manufactured by the method of claim 1.

8. A polar phenolic extract of EVOO comprising EVOO that has been extracted with an ethanol/water solution than then has been washed with a solution of heptane.
